(19) United States
(12) Patent Application Publication
(10) Pub. No.: US 2015/0290251 A1
(43) Pub. Date: Oct. 15, 2015

(54) USE OF EGGSHELL MEMBRANE FORMULATIONS TO ALLEVIATE JOINT PAIN

(71) Applicant: U.S. NUTRACEUTICALS, LLC d/b/a Valensa International, EUSTIS, FL (US)

(72) Inventors: JOHN A. MINATELLI, MOUNT DORA, FL (US); W. STEPHEN HILL, OCALA, FL (US); MICHAEL CIELENSKY, WESTON, FL (US)

(73) Assignee: U.S. NUTRACEUTICALS, LLC d/b/a Valensa International

(21) Appl. No.: 14/748,285

(22) Filed: Jun. 24, 2015

Related U.S. Application Data
(62) Division of application No. 12/946,924, filed on Nov. 16, 2010.
(60) Provisional application No. 61/261,921, filed on Nov. 17, 2009.

(51) Int. Cl. A61K 35/57; A61K 31/728; A61K 31/19; A61K 38/39; A61K 36/324; A61K 31/122; A61K 45/06

(52) U.S. Cl. CPC A61K 35/57; A61K 31/122; A61K 31/728; A61K 45/06; A61K 38/39; A61K 36/324; A61K 31/19

(57) ABSTRACT

A method to treat and alleviate symptoms of joint pain in an individual is accomplished by administering a therapeutic amount of fowl eggshell membrane or processed eggshell membrane preparations in synergistic combination with other active constituents in an oral dosage form.

### RELATED APPLICATION

This application is a divisional of U.S. application Ser. No. 12/946,924 filed on Nov. 16, 2010, which is based upon U.S. provisional application Ser. No. 61/261,921 filed Nov. 17, 2009, the disclosures which are hereby incorporated by reference in their entirety.

### FIELD OF THE INVENTION

This invention relates to eggshell membrane applications, and more particularly, this invention relates to treating and alleviating symptoms of joint pain using eggshell membranes or processed eggshell membrane preparations.

### BACKGROUND OF THE INVENTION

It is known to use eggshell membrane compositions to alleviate everyday joint aches and pains, reduce joint discomfort, and improve joint mobility, flexibility and function. These eggshell membrane preparations are used to promote a normal inflammatory response and improve motion and provide some antioxidant activity.

U.S. Patent Publication Nos. 2008/0234195 and 2009/0104173 and U.S. Pat. No. 6,946,551 disclose various compositions derived from eggshell membrane that include hyaluronic acid in combination with naturally occurring constituents derived from eggshell membrane that can be used in one aspect for treating joint pain and joint problems. There are also compounds such as BiovaFlex™ as a natural joint health ingredient, which includes proteins and peptides derived from hydrolyzed, water-soluble egg membrane used for joint treatment.

### SUMMARY OF THE INVENTION

A method to treat and alleviate symptoms of joint pain in an individual is accomplished by administering a therapeutic amount of fowl eggshell membrane or processed eggshell membrane preparations in synergistic combination with other active constituents in an oral dosage form. A composition is also disclosed.

### DETAILED DESCRIPTION

The present invention will now be described more fully hereinafter. This invention may, however, be embodied in many different forms and should not be construed as limited to the embodiments set forth herein. Rather, these embodiments are provided so that this disclosure will be thorough and complete, and will fully convey the scope of the invention to those skilled in the art.

A method to treat and alleviate symptoms of joint pain in an individual is disclosed and includes administering a therapeutic amount of fowl eggshell membrane or processed eggshell membrane preparations in synergistic combination with other active constituents in an oral dosage form. The eggshell membrane preparation in one example has been processed to increase water solubility or enriched to increased concentration of active substances. In another example, the formulation delivers 0.5-1000 mg of eggshell membrane or processed eggshell membrane preparation per daily dose. The preferred formulation delivers 40-500 mg eggshell membrane or processed eggshell membrane preparation per daily dose.

In yet another aspect, the formulation is supplemented with various molecular weight polymers of hyaluronic acid or sodium hyaluronate (hyaluronan) derived from microbial fermentation, eggshell or animal tissue whether in pure or crude form. For example, the formulation delivers 50-1000 mg hyaluronan per daily dose. In another aspect, hyaluronic acid is preferably derived from a biofermentation process and has a molecular weight between 0.5 and 100 kilodaltons (kDa). The hyaluronic acid has a molecular weight greater than 100 kDa in another aspect.

Astaxanthin is added in another aspect and has synergistic effects in combination with the other ingredients. The formulation is supplemented to deliver 0.1-12 mg astaxanthin per daily dose in one example. The astaxanthin is derived from Haematococcus pluvialis algae, Pfaffia, krill, or by synthetic routes, in the free diol, monoester or diester form in another example.

A natural or synthetic cyclooxygenase-1 or -2 inhibitor such as aspirin, acetaminophen, steroids, prednisone, NSAIDs, turmeric, Curcumin, boswellia and the like is added in another example. The composition also includes a gamma-linoleic acid rich oil such as from Borage (Borago officinalis L.), Safflower (Carthamus tinctorius L.) and the like, and is combined with an n-3 (omega-3) fatty acid rich oil such as from: fish oil, algae oil, flax seed oil, soybean oil, perilla seed oil, chia seed oil and the like—with the n-3 fatty acid being alpha-linolenic, stearidonic, eicosapentaenoic or docosapentaenoic acid. The composition is supplemented with collagen in any of its various forms in one example.

In another example, the composition is combined with anti-inflammatory and/or joint health promoting compounds such as preparations of: green lipped mussel (Perna canaliculus), Boswellia serrata, turmeric (Curcuma longa), stinging nettle (Urtica dioica), Andrographis, Cat's claw (Uncaria tomentosa), white willow (Salix alba), bromelain, Vitamin D, Magnesium, milk protein concentrates, fatty acid esters, methylsulfonylmethane (MSM), chondroitin sulfate, glucosamine sulfate, glucosamine hydrochloride, s-adenosyl methionine, proanthocyanidins, procyanidins or flavonoids. The ingredients are formulated in a chewable dosage form in another example, which could include a tablet or “gummy.”

In yet another example, a pharmaceutically acceptable formulation of eggshell membrane or processed eggshell membrane preparation is combined or supplemented with at least one of the following: glucosamine sulfate, chondroitin sulfate, collagen, astaxanthin, hyaluronic acid, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

In still another example, a dietary supplement acceptable formulation of eggshell membrane or processed eggshell membrane preparation is combined or supplemented with at least one of the following: glucosamine sulfate, chondroitin sulfate, collagen, astaxanthin, hyaluronic acid, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

In another example, a medical food acceptable formulation of eggshell membrane or processed eggshell membrane preparation is combined or supplemented with at least one of the following: glucosamine sulfate, chondroitin sulfate, collagen, astaxanthin, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

As to astaxanthin, it is beneficial and synergistic in combination with the other components, and as noted in the commonly assigned U.S. Patent Publication No. 2008/0124391, the disclosure which is hereby incorporated by reference in its entirety, astaxanthin (3,3′-dihydroxy-β, β-carotene-4, 4′dione) CAS [471-53-4], is a keto carotenoid pigment naturally accumulated via the diet in marine animals such as salmon, shrimp, red seabream and lobster and in birds such as flamingoes. Astaxanthin also occurs in certain microalgae such as Haematococcus pluvialis and in yeasts such as Phaffia species. The highest concentration, up to four percent of dry matter, occurs in Haematococcus. It can be chemically synthesized and obtained in naturally occurring stereoisomer form.

Astaxanthin, although related to other carotenoids such as beta-carotene, zeaxanthin and lutein, is a more powerful antioxidant. Astaxanthin is particularly potent in quenching singlet oxygen and has over five hundred times the ability to quench singlet oxygen as alpha-tocopherol. This antioxidant activity of astaxanthin is thought to be responsible for the wide range of health-promoting properties it exhibits, including skin and eye protection from damage by UV-light, anti-inflammatory activity, modulation or promotion of the immune response, reduction in aging processes and benefits to heart, liver, joints and prostate.

Astaxanthin is a pigment imparting the pinkish-red hue to the flesh of salmon and trout, and the coloring in the carapaces of shrimp, lobsters and crayfish. The astaxanthin molecule has two asymmetric carbons located at the 3 and 3′ positions of the cyclohexenone rings on either end of the molecule. Different enantiomers of the molecule result from how the hydroxyl groups (—OH) are attached to the carbon atoms at these centers of asymmetry. The three possible enantiomers of astaxanthin are (3R,3′R), (3S,3′S) and (3R,3′S; meso).

Free astaxanthin and its mono- and diesters from Haematococcus have optically pure (3S,3′S)-chirality. The (3S,3′S) isomer of astaxanthin is found in the skin and flesh of some salmonid fish.

Natural astaxanthin extract is an oily, viscous dark red lipophilic extract. The extract contains free astaxanthin, astaxanthin fatty acid mono-esters and astaxanthin fatty acid di-esters along with triglycerides and other lipophilic compounds. Carotenoid pigments from different sources of Haematococcus pluvialis include in some examples astaxanthin (total) 81-99% (which comprises free astaxanthin 1-5%; astaxanthin monoesters 46-79%; astaxanthin diesters 10-39%); 3-carotene 0-5%; lutein 1-11%; canthaxanthin 0-5.5%; and other carotenoids 1-9%.

Naturally derived astaxanthin is typically the 35,3′S stereoisomer found in Haematococcus algae or 3R,3R′, primarily found in Phaffia yeast. Synthetic astaxanthin has a more complex stereoisomeric profile due to the non-stereo selectivity from the reaction conditions used in its manufacture. Haematococcus pluvialis contains mono and diesterified astaxanthin as the predominant forms of astaxanthin, while Phaffia and synthetically produced astaxanthin lack these esterifications.

Natural astaxanthin extracts contain astaxanthin in E and Z isomeric configurations.

Natural astaxanthin extract derived from Haematococcus pluvialis as an example includes astaxanthin stereoisomers as follows: (3S,3′S) 100%; (3S,3′R) and (3R,3′S) 0%; (3R,3′R) 0%, with the geometric isomer proportions, expressed as a percentage of the total astaxanthin, of about: E-astaxanthin 59%; 9Z-astaxanthin 15%; 13Z-astxanthin 26%, and non-astaxanthin carotenoid levels of about: 0.3% β-carotene, 0.07% lutein, 0.3% canthaxanthin and 1.3% total other carotenoids.

It includes low molecular weight hyaluronic acid (HA) that is a major hydrodynamic component of synovial fluid and is a natural absorbent and lubricant for bones. It is excellent for bioavailability and maximizes interaction with target synovial cells and has anti-inflammatory mechanisms and down regulates pro-inflammatory mediators and neuro-peptide production and improves the symptoms of osteoarthritis. The HA's with molecular weights within the range of 0.5 to about 1 times 10⁶ DA were generally more effective in reducing indices of synovial inflammation and storing rheological properties of SF (visco-induction) than HA's with molecular weight greater than 2.3 times 10⁶ DA.

In induced uveitis, astaxanthin showed dose dependent ocular anti-inflammatory activity by suppression of NO, PGE-2 and TNF-Alpha by directly blocking NO synthase activity. It reduces C-Reactive Protein (CRP) blood levels: In human subjects with high risk levels of CRP three months of astaxanthin treatment resulted in 43% of patients serum CRP levels to drop below the risk level. Astaxanthin is so powerful it completely negates the pro-oxidant activity of Vioxx, which is known to cause cellular membrane lipid peroxidation leading to heart attack and stroke. Astaxanthin is absorbed by in vitro lens epithelial cells where it dramatically suppresses UVB induced lipid peroxidative mediated cell damage at umol/L concentrations suggesting use for the prevention of cataracts. In human trials astaxanthin at 4 mgs/day completely prevented joint pain following strenuous knee exercise when compared to untreated subjects.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that the modifications and embodiments are intended to be included within the scope of the dependent claims.

That which is claimed is:

1. A dietary supplement composition formulated in a therapeutic amount to treat and alleviate symptoms of joint pain in an individual, comprising a soluble eggshell membrane preparation in combination with astaxanthin and low molecular weight hyaluronic acid or sodium hyaluronate (hyaluronan) having a molecular weight of between 0.5 and 100 kilodaltons (kDa) in an oral dosage form.

2. The composition according to claim 1, comprising 0.5 to 1,000 mg of soluble eggshell membrane preparation delivered per daily dose.

3. The composition according to claim 1, comprising 40 to 500 mg of soluble eggshell membrane preparation delivered per daily dose.

4. The composition according to claim 1, wherein the low molecular weight hyaluronic acid or sodium hyaluronate (hyaluronan) is derived from microbial fermentation.

5. The composition according to claim 1, comprising 50 to 1,000 mg hyaluronan delivered per daily dose.

6. The composition according to claim 1, comprising 0.1 to 12 mg astaxanthin delivered per daily dose.

7. The composition according to claim 6, wherein the astaxanthin is derived from Haematococcus pluvialis algae, Pfaffia, krill, or by synthetic routes, in the free diol, monoester or diester form.

8. The composition according to claim 1, comprising a natural or synthetic cyclooxygenase-1 or -2 inhibitor comprising aspirin, acetaminophen, steroids, prednisone, or NSAIDs.

9. The composition according to claim 1, comprising collagen.

10. The composition according to claim 1, comprising anti-inflammatory and/or joint health promoting compounds comprising at least one of green lipped mussel (Perna canaliculus), Boswellia serrata, turmeric (Curcuma longa), stinging nettle (Urtica dioica), Andrographis, Cat's claw (Uncaria tomentosa), white willow (Salix alba), bromelain, Vitamin D, Magnesium, milk protein concentrates, fatty acid esters, methylsulfonylmethane (MSM), chondroitin sulfate, glucosamine sulfate, glucosamine hydrochloride, and s-adenosyl methionine.

11. The composition according to claim 1, wherein the composition is formulated in a chewable dosage form such as a tablet, gelatin or pectin based “gummy.”

12. A dietary supplement composition comprising a therapeutically effective amount of a fowl eggshell membrane in combination with hyaluronic acid having a molecular weight between 0.5 and 100 kilodaltons (kDa) and astaxanthin in an oral dosage form for use in the treatment of joint pain in an individual.

13. The composition according to claim 12, wherein said eggshell membrane comprises an eggshell membrane preparation that has been enriched to increased concentration of active substances.

14. The composition according to claim 12, wherein the composition delivers 0,5 to 1,000 mg of fowl eggshell membrane per daily dose.

15. The composition according to claim 12, wherein the composition delivers 40 to 500 mg of fowl eggshell membrane per daily dose.

16. The composition according to claim 12, wherein the hyaluronic acid is derived from microbial fermentation, eggshell or animal tissue in pure or crude form.

17. The composition according to claim 12, wherein the composition delivers 0.1 to 12 mg astaxanthin per daily dose.

18. The composition according to claim 17, wherein the astaxanthin is derived from Haematococcus pluvialis algae, Pfaffia, krill, or by synthetic routes, in the free diol, monoester or diester form.

19. The composition according to claim 12, and further comprising a natural or synthetic cyclooxygenase-1 or -2 inhibitor including at least one of aspirin, acetaminophen, steroids, prednisone, and NSAIDs.

20. The composition according to claim 12, and further comprising an n-3 (omega-3) fatty acid rich oil derived from fish oil, algae oil, flax seed oil, soybean oil, perilla seed oil, and chia seed oil, wherein the n-3 fatty acid comprises at least one of alpha-linolenic, stearidonic, eicosapentaenoic or docosapentaenoic acid.

21. The composition according to claim 12, and further comprising at least one of an anti-inflammatory and joint health promoting compound derived from at least one of green lipped mussel (Perna canaliculus), Boswellia serrata, turmeric (Curcuma longa), stinging nettle (Urtica dioica), Andrographis, Cat's claw (Uncaria tomentosa), white willow (Salix alba), bromelain, Vitamin D, Magnesium, milk protein concentrates, fatty acid esters, methylsulfonylmethane (MSM), chondroitin sulfate, glucosamine sulfate, glucosamine hydrochloride, and s-adenosyl methionine.

22. A dietary supplement composition consisting of 100 to 500 mg of hydrolyzed fowl eggshell membrane preparation, 10 to 40 mg sodium hyaluronate that is less than 100 kDa, 0.5 to 12 mg astaxanthin, Boswellia serrata preparation providing at least 30 to 100 mg Boswellic acids.
